(19) United States
(12) Patent Application Publication
(10) Pub. No.: US 2007/0184058 A1
(43) Pub. Date: Aug. 9, 2007

(54) Glucocerebroside treatment of pulmonary or respiratory diseases or disorders

(76) Inventors: Yaron Ilan, Givat Massua (IL); Gadi Lalazar, (US)

Correspondence Address: ENZO BIOCHEM, INC., 527 MADISON AVENUE (9TH FLOOR), NEW YORK, NY, 10022, US

(21) Appl. No.: 11/591,153

(22) Filed: Nov. 1, 2006

Related U.S. Application Data
(63) Continuation-in-part of application No. 11/378,941, filed on Mar. 17, 2006.
Application No. 11/378,941 is a continuation-in-part of application No. 10/675,980, filed on Sep. 30, 2003.
Application No. 10/675,980 is a continuation-in-part of application No. 10/375,906, filed on Feb. 27, 2003.

(51) Int. Cl. A61K 39/00; A61K 39/12; A61K 39/38

(52) U.S. Cl. 424/184.1; 424/204.1

(57) ABSTRACT

This invention relates to the use of naturally occurring mammalian intermediary metabolites, Tcell ligands or Tcell receptor ligands, preferably glycosylceramides, for the treatment or prevention of immune mediated or immune related diseases or disorders. Specifically, the present invention provides compositions and methods for the treatment or prevention of pulmonary, respiratory or airway diseases or disorders such as asthma.

### REFERENCE TO RELATED PATENT APPLICATIONS

This application is a continuation-in-part of U.S. patent application Ser. No. 11/378,941, filed on Mar. 17, 2006, entitled “Glucocerebroside Treatment of Liver Disorders,” which is a continuation-in-part of U.S. patent application Ser. No. 10/675,980, filed on Sep. 30, 2003, entitled “Glucocerebroside Treatment of Disease,” which is a continuation-in-part of U.S. patent application Ser. No. 10/375,906, filed on Feb. 27, 2003, entitled “Regulation of Immune. Responses by Manipulation of Intermediary Metabolite Levels.” The contents of the aforementioned patent applications are hereby incorporated by reference, in their entireties.

### FIELD OF THE INVENTION

This invention relates to the use of a naturally occurring, mammalian intermediary metabolites or T cell receptor ligands, preferably a glycosylceramide, for the treatment or prevention of immune mediated or immune related diseases or disorders. Specifically, the present invention provides compositions and methods for the treatment or prevention of pulmonary, respiratory or airway diseases or disorders such as asthma.

All patents, patent applications, patent publications, scientific articles, and the like, are hereby incorporated by reference in their entirety in order to describe more fully the state of the art to which the present invention pertains.

### BACKGROUND OF THE INVENTION

Various methods have been described for the treatment of immune related or immune mediated disorders or diseases, infectious diseases, metabolic disorders and different types of cancer in mammalian subjects. One of these methods involves the modulation of immune responses in a subject. This includes the down regulation of the immune response system using procedures or combinations of procedures for producing and applying a new and unexpected immune modulation termed selective immune down regulation (SIDR). Immunological modulation is an artificially induced variation in a subject's immune system in response to the introduction of reagents, procedures and processes. These procedures have been described in detail in U.S. patent application Ser. No. 08/808,629, filed on Feb. 28, 1997, U.S. patent application Ser. No. 10/377,628, filed on Mar. 4, 2003, U.S. application Ser. No. 10/377,603, filed on Mar. 4, 2003, U.S. patent application Ser. No. 09/447,704, filed on Feb. 28, 1997, U.S. application Ser. No. 10/385,440, filed on May 9, 2001, and U.S. application Ser. No. 09/356,294, filed on Jul. 16, 1999. Each of the foregoing patents is incorporated by reference in its entirety in the present application and may further be used in conjunction with the present invention.

Other methods describe the use of educated or treated cells in the treatment of a variety of diseases. Specifically, the methods are directed to the manipulation of the NKT cell population in a subject that results in the modulation of the Th1/Th2 balance toward anti-inflammatory or pro-inflammatory cytokine producing cells. A detailed description of these inventions have been disclosed in U.S. patent application Ser. No. 10/451,811, entitled “Educated NKT Cells and Their Uses in the Treatment of Immune-Related Disorders” by Yaron Ilan et al., filed on Jun. 25, 2003, PCT Application No. IL01/01197, filed on Dec. 24, 2001, and U.S. application Ser. No. 10/375,906, filed on Feb. 27, 2003. Each of the foregoing patents is incorporated by reference in its entirety in the present application and may further be used in conjunction with the present invention.

The present invention provides a new method for the treatment or prevention of pulmonary, respiratory and airway diseases or disorders such as asthma, in mammalian subjects, and preferably, in human subjects. This method involves the administration of a mammalian intermediary metabolite to a subject. In a preferred embodiment the mammalian intermediary metabolite is a T cell ligand or a T cell receptor ligand.

The mammalian intermediary metabolite, the T cell ligand or the T cell receptor ligand may comprise a lipid, a polar lipid, or a conjugated biomolecule. The conjugated biomolecule may in turn comprise a glycolipid, a sulfated glycolipid, a lipoprotein, an apolipoprotein, a glycoprotein other than an antibody, a cytokine, or a hormone. A glycolipid may comprise a monosaccharide ceramide, a disaccharide ceramide or a polysaccharide ceramide, with a β-linkage between the saccharide and ceramide portions. Examples of β-linked monosaccharide ceramides can include but not be limited to a glucosylceramide, or a galactosylceramide and an example of a disaccharide ceramide can include but not be limited to lactosylceramide. It is also understood that any derivatives of the foregoing as well as any analogs other than α-linked analogs may also find use.

Glucosylceramide is a naturally occurring glycolipid consisting of ceramide, to which glucose is attached. A ceramide, which is a sphingosine and a fatty acid, is the structural unit common to all sphingolipids. Sphingolipids have a variety of cellular functions. These include membrane structural roles and cell signaling participation. (Sullard et al., 2000 Journal of Mass Spectrometry 35: 347-353.) Glucosylceramide is made by the enzyme glucosylceramide synthase which attaches the two molecules together. (see FIG. 1 and FIG. 2). An example of a glucosylceramide includes glucocerebroside, or a glucocerebroside analog or derivative.

An example of a pulmonary, respiratory or airway disease or disorder is asthma. Asthma is a respiratory disease caused by an inappropriate response to usually innocuous environmental stimuli. Exposure to the stimulant can lead to a series of adverse reactions such as inflammation of the bronchial passages, increased levels of mucus secretions and difficulty in breathing. Cellular markers for asthma include increased levels of eosinophils, CD+T4 lymphocytes and IgE producing B cells. Cytokine markers can include increased levels of IL4, IL5 and IL13. Although the exact pathway that leads to asthma remains unknown, induction of the allergenic response in asthma seems to involve a participatory role of NKT cells, since mutants lacking this class of T-cell do not develop airway hyper-reactivity in model systems (Lisbonne et al., 2003 J. Immunol. 171; 1637-1641, Akbari et al., 2003 Nat. Med. 9; 582-588).

Knowledge of a role of NKT cells in this system has led to the investigation of potential effects by α-galactosylceramide, since it is well known that this compound can induce dramatic effects upon NKT cells. Treatment of animals with α-galactosylceramides was first thought to produce a killing effect upon NKT cells since shortly after its administration, they seemed to completely disappear from the circulatory system (Matsuda et al., 2000 J. Exp Med, 192; 741-754 and Fujii et al., 2002 Nat Immunol 3; 867-874). It was later discovered that this effect was not a selective lethality effect upon NKT cells but rather that there was a loss of the marker used to identify these cells. When a different marker was used, the NKT cells were seen to still be present and furthermore, in a reversal of the earlier conclusions, there was stimulation and expansion of the NKT population after exposure to α-galactosylceramide (Crowe et al., 2003 J Immunol, 171; 4020-4027 and Wilson et al., 2003 Proc Nat Acad Sci USA 100; 10,913-10,918).

Effects of α-galactosylceramide on the treatment of disease have been described in a number of different systems. For example, α-galactosylceramide has been used for enhancing immune responses as a treatment for anti-tumor and anti-pathogen activities and it has also been used to quell deleterious immune responses that are observed in diabetes and experimental autoimmune encephalomyelitis (Furlan et al., 2003 Eur J Immunol 33; 1830-1838 and Singh et al., 2001 J Exp Med 194; 1801-1811).

Since NKT cells had previously been implicated in the development of asthma and α-galactosylceramide has been shown to be an effective immune modulator of NKT activity, α-galactosylceramide was investigated as a candidate for the treatment of asthma in a number of different laboratories. The results of these studies demonstrated strong effects upon animal models of asthma but a layer of complexity was revealed where it was found that there could be profoundly different effects, i.e. administration of α-galactosylceramide gave relief in some situations and exacerbated deleterious effects in other circumstances. Some understanding of these conflicting results may be achieved by dividing the series of experiments into two categories:

1) test animals that were never exposed to an allergen prior to administration of α-galactosylceramide; and

2) test animals that were sensitized to a particular allergen and given α-galactosylceramide afterwards.

In a series of experiments where animals were sensitized to Ovalbumen (OVA), a surrogate for an asthma allergen, and then later challenged with nasal administration of OVA, the administration of α-galactosylcerebroside to the test animals either 2 hours prior to or during the course of the sensitization procedure (the first group format) led to exacerbation of the markers, signs and symptoms for asthma when the animals were later presented with the OVA challenge (Bilenki et al., 2004 Eur J Immunol. 34; 345-354, Kim et al., 2004 J. Allerg Clin Immunol 114; 1332-1338, Morishima et al., 2005 Eur J Immunol 35; 2803-2814). In fact, in one of these references (Kim et al., 2004) there was no reactivity to the challenge unless α-galactosylcerebroside accompanied the OVA during the sensitization step. Indeed, it was found that the intranasal (but not intravenous) administration of the α-galactocylcerebroside itself to otherwise naïve animals could lead to asthma like symptoms (Meyer et al., 2006 Proc Nat Acad Sci USA 103; 2782-2787). Under these circumstances it appears that α-galactocylcerebroside acted as an adjuvant in enhancing an immune response to an allergen. This has been previously seen in other systems where α-galactosylcerebroside has been used as an adjuvant in immunization procedures for malaria vaccines (Gonzales-Aseguinolaza et al., 2002 J Exp. Med. 195; 617-624) as well as immunization against influenza and adenovirus (Ko et al., 2005 J. Immunol 175; 3309-3317).

In a series of experiments from the second group format, where sensitization of animals with OVA was carried out first and at a later time α-galactosylcerebroside was administered at the time of the challenge with OVA, the results were very different form the first group. Under these circumstances, α-galactosylcerebroside acted more like a toleragen, reducing the severity of the response to the allergen (Morishima, 2005 op. cit., Hachem et al., 2005 Eur J Immunol 35; 2793-2802 and Matsuda et al., 2005 Am J. Respir. Cell Mol. Biol. 31; 22-31). It would seem that the alteration of the directionality of the α-galactosylcerebroside effect from the first group may be due to an inflammatory response already being established in the test animal at the time of the α-galactosylcerebroside administration. This viewpoint is also supported by the results with administration of α-galactosylcerebroside alone. As described above, administration of this compound led to induction of asthma like symptoms, but when this experiment was continued and a second dose of was administered, the induction of asthma-like symptoms was strongly reduced (Meyers, 2005 op. cit.). This is potentially a demonstration of the dual effects of α-galactosylcerebroside acting as an immunogen in the first administration and then acting as a toleragen in the second administration when an immune response had already been established.

On the other hand, this particular compound has been shown to have deleterious side effects that may counterbalance its potential application to therapeutic treatment of asthma. For instance, α-galactosylcerebroside has been shown to induce hepatic damage (Osman et al., 2000 Eur J Immunol 30; 1919-1928, Nakagawa et al., 2001 J. Immunol 166; 6578-6584).

### SUMMARY OF THE INVENTION

This invention relates to the use of a naturally occurring mammalian intermediary metabolite, for the treatment or prevention of pulmonary, respiratory or airway diseases or disorders in mammalian subjects. In a preferred embodiment, the disease being treated or prevented is asthma and the mammalian intermediary metabolite is a T cell ligand or T cell receptor ligand.

This invention further provides a process for treating or preventing pulmonary, respiratory or airway diseases or disorders in a mammalian subject comprising administering to said subject an effective amount of a mammalian intermediary metabolite, wherein said metabolite is a T cell ligand, a T cell receptor ligand, a lipid, a polar lipid, a conjugated biomolecule, a glycolipid, a sulfated glycolipid, a lipoprotein, an apolipoprotein, a glycoprotein other than an antibody, a cytokine, a hormone, a monosaccharide ceramide, a disaccharide ceramide, a polysaccharide ceramide, a non-α-linked glycosylceramide, a β-glycolipid, a β-glycolipid derivative, an analog of a β-linked glycolipid other than an α-linked glycolipid, β-glycosylceramide, a β-glycosylceramide derivative or an analog of a β-linked glycosylceramide other than an α-linked glycosylceramide.

Another aspect of the present invention provides for the treatment or prevention of a disease or disorder in a mammalian subject comprising the ex vivo treating or educating of cells obtained from the mammalian subject. The cells are treated or educated with an effective amount of an intermediary metabolite, wherein said metabolite is a T cell ligand, a T cell receptor ligand, a lipid, a polar lipid, a conjugated biomolecule, a glycolipid, a sulfated glycolipid, a lipoprotein, an apolipoprotein, a glycoprotein other than an antibody, a cytokine, a hormone, a monosaccharide ceramide, a disaccharide ceramide, a polysaccharide ceramide, an α-glycosylceramide, a non-α-linked glycosylcerebroside, a β-glycolipid, a β-glycolipid derivative, an analog of a β-linked glycolipid other than an α-linked glycolipid, β-glycosylceramide, β-glycosylceramide derivative or an analog of a β-linked glycosylceramide other than an α-linked glycosylceramide. The treated or educated cells are then re-administered to the subject.

The present invention also relates to the treatment or prevention of a disease in a mammalian subject comprising the re-administration of treated or educated cells to the subject, and the direct administration to said subject of an effective amount of an intermediary metabolite, wherein said metabolite is a T cell ligand, a T cell receptor ligand, a lipid, a polar lipid, a conjugated biomolecule, a glycolipid, a sulfated glycolipid, a lipoprotein, an apolipoprotein, a glycoprotein other than antibodies an antibody, a cytokine, a hormone, a monosaccharide ceramide, a disaccharide ceramide, a polysaccharide ceramide, an α-glycosylcerebroside, a non-α-linked glycosylcerebroside, a β-glycolipid, a β-glycolipid derivative, an analog of a β-linked glycolipid, β-glycosylceramide, β-glycosylceramide derivative or an analog of a β-linked glycosylceramide.

Numerous other aspects and embodiments of the present invention are described in further detail below.

### DETAILED DESCRIPTION OF THE INVENTION

The present invention provides methods for the treatment or prevention of a pulmonary, respiratory or airway disease or disorder in a mammalian subject by the administration of an effective amount of a mammalian intermediary metabolite to a subject. The mammalian intermediary metabolite includes, but is not limited to a T cell ligand, a T cell receptor ligand, a lipid, a polar lipid, a conjugated biomolecule, a glycolipid, a sulfated glycolipid, a lipoprotein, an apolipoprotein, a glycoprotein other than an antibody, a cytokine, a hormone, a monosaccharide ceramide, a disaccharide ceramide, a polysaccharide ceramide, a glucosylceramide, a galactosylceramide, a glycosylceramide, a glycosylceramide derivative, a glycosylceramide analog other than an α-linked glycosylceramide, a sphingosine, a sphingolipid, a ceramide, a β-glucosylceramide, a β-glucosylceramide derivative, a β-glucosylceramide analog other than an α-linked glucosylceramide a β-galactosylceramide, a β-galactosylceramide derivative, a β-galactosylceramide analog other than an α-linked galactosylceramide, a β-lactosylceramide, a β-lactosylceramide derivative, or a β-lactosylceramide analog other than an α-linked lactosylceramide. In a preferred embodiment of the invention, the mammalian subject is a human being.

In a preferred embodiment of the invention, the mammalian subject is a human being, the pulmonary and/or respiratory disease is asthma, and the mammalian intermediary metabolite is a β-glycosylceramide,

The β-glycosylceramide and its analogs or derivatives may be administered by oral, intravenous, intraperitoneal, intramuscular, parenteral, transdermal, intravaginal, intranasal, mucosal, sublingual, topical, rectal or subcutaneous administration, by inhalation or any combination thereof. It has already been disclosed in related applications that this class of mammalian intermediary metabolites has dual properties. The administration of β-glucosylceramide can enhance immune surveillance as shown in studies with dietetic augmentation where its inclusion in food resulted in suppression of tumor growth with chemically treated (Schmelz et al., 1999) and genetically tumor-prone mice (Symolon et al., 2003). On the other hand, β-glycosylceramides can have immune suppressive abilities in situations where immune responses are actually disadvantageous to the test animal. An example of this beneficial effect has been seen with treatment of conA-induced hepatitis (Margalit et al., 2005 Am J Physiol Gastroint Liver Physiology 289; G917-G925).

This duality of potentiation has previously been seen with α-galactosylceramide and we have previously discussed this in the context of treating animal models for asthma where one format led to exacerbation of a reaction and a second format led to alleviation of symptoms. In terms of translating the results of animal model laboratory experiments with α-galactosylceramide into therapies for asthma patients, the second group format is more reflective of potential protocols for amelioration of asthma and its symptoms. In common with the second group of animal models, sensitization to some environmental antigens has already taken place in these individuals and it is because an immune response has already been induced that places them in need of a therapeutic intervention to alleviate symptoms. Thus, it is apparent that the hypersensitivity to these antigens that is already induced could allow α-galactosylceramide to provide beneficial results when administered to asthma patients.

However, this substance is foreign to mammalian cells and as such, its interactions with mammalian components are of an artificial nature. Furthermore, as described previously, it can induce asthma on its own. Additionally, this compound is known to induce hepatic cell damage. Thus some embodiments of the present invention avoid the use of α-galactosylceramide and use β-glycosylceramides, thereby permitting appropriate immunomodulatory effects on asthma while avoiding consequences of a foreign appearing compound.

The sugar group of the glycosylceramides can be a monosaccharide such as glucose or lactose (glycosylceramides and lactosylceramides) or a disaccharide such as galactose (galactosylceramides). Intermediary metabolites such as glycosylceramides can be purified and isolated from natural sources or they can be synthesized artificially.

On the one hand, purification from a natural source allows an inexpensive source of the reagent to be used for the present invention. Also, it should be pointed out that numerous biological molecules that are mammalian intermediary metabolites are synthesized in other biological systems besides mammalian cells. Thus, the same identical molecule may be found in mammalian cells, non-mammalian eukaryotic cells and even in prokaryotes such as bacteria or yeast. In principle, these non-mammalian sources may also be used to provide desirable intermediary metabolites. As such, in the present invention, an intermediary metabolite is defined as a mammalian intermediary metabolite strictly in terms of whether it is a molecule that is naturally present in a mammalian cell and not the particular source from which it is isolated in order to be used in a therapeutic procedure. However, one drawback of the use of a biological systems approach is that these natural sources of glycosylceramides frequently consist of a large family of similar species that vary in the length of their carbon chains and placements of double bonds. Thus isolation of a single species may be problematic with some sources. On the other hand, some sources such as soy beans display only a single species, thereby allowing almost a pre-purified supply.

On the other hand, directed synthesis of a particular glycosylceramide offers the advantage that no mammalian or other cells are needed and a series of synthetic steps should culminate in a single specific species of β-glycosylceramide. Side products of the various reactions in these steps should usually be chemically differentiated enough that the desired products will be readily separated, leading to a final product with a selected length for each carbon chain as well as the presence of a double bond at any desired site in the chains. The use of synthetic routes will also allow the use of glycosylceramide analogs in the present invention, where substitutions may be used for various components. An example of this approach would be synthesis of an analog where the oxygen joining the sugar to the ceramide portion is replaced by a carbon or sulfur atom.

A third route is also possible that combines synthetic and natural sources, where a particular desired intermediary metabolite is present in a mammalian cells but there are no convenient alternative non-mammalian sources. In this case one or more of the genes in the pathway for synthesis of the desired intermediary metabolite can be cloned and inserted into a bacterial or yeast expression vector. As long as there is sufficient amount of an appropriate precursor in the host cells, the vector can allow the production of the desired intermediary mammalian metabolite in a non-mammalian host.

The present invention describes a method for treating a disease where regulatory, immune-regulatory or NKT cells are obtained from the subject to be treated, or from another subject, and are educated or treated ex vivo. The cells are treated or educated by the presence of intermediary metabolite, antigens or epitopes, and antigen presenting cells, or any combination thereof. The treated or educated cells are then re-administered to the subject. The cells may be administered to the subject by adoptive transfer.

In addition to the method described above involving the ex vivo treatment or education of cells, the present invention also provides for a method where the ex vivo treatment or education is accompanied by the method of directly administering to the subject to be treated, by a variety of ways, an effective amount of the intermediary metabolite, antigen presenting cells, and antigens or epitopes, or any combination of the above. The disease may also be treated by only the direct administration of an effective amount of the intermediary metabolite, antigen presenting cells, and antigens or epitopes, or any combination of the above.

A therapeutic composition for the use in the treatment of the disease may comprise an effective amount of the intermediary metabolite, antigen presenting cells, and antigens or epitopes, or any combination of the above.

The treatment of a disease in any of the described methods results in a change in the number or function of regulatory, immune-regulatory or NKT cells. This change encompasses a reduction, inhibition, or decrease in the number or function of the cells. This inhibition may be caused by the competitive displacement of activating elements from the CD1d molecule. A change may also include a stimulation or increase in the number or function of the cells. This stimulation may be caused by increased binding of the activating elements from the CD1d molecule.

The treatment of a disease may also result in a change in the cytokine responses. Any cytokine in the immune system may be involved in these responses. The change could result in a pro-inflammatory or an anti-inflammatory response. There may also be a pro-inflammatory, and an anti-inflammatory response since certain cytokines may increase and others may decrease, simultaneously.

Another result of the treatment of a disease is an alteration of the regulatory, immune-regulatory or NKT cell distribution in the subject. This change may also be accompanied by a change in the peripheral/intrahepatic T cell ratio. A further result may also include a change in intrahepatic CD8+ T cell trapping. There may be an increase or a decrease in the intrahepatic trapping. The result may also include a change in intrasplenic T cell trapping, where said change could be an increase or decrease.

Also provided in the present invention are two in vitro screening assays for an analog or derivative of an intermediary metabolite which is administered to the subject to treat a disease. The first assay involves providing regulatory, immune-regulatory or NKT cells from the subject being treated or another subject, antigen presenting cells, and an analog or derivative of the intermediary metabolite in vitro. If a decrease in the regulatory, immune-regulatory or NKT cell proliferation is identified, then that specific analog or derivative is a treatment for disease.

The second assay involves providing in a first test tube, regulatory, immune-regulatory or NKT cells and BSA; in a second test tube, regulatory, immune-regulatory or NKT cells and the analog or derivative of an intermediary metabolite; in a third test tube, regulatory, immune-regulatory or NKT cells, antigen presenting cells and BSA; and in a fourth test tube, regulatory, immune-regulatory or NKT cells, antigen presenting cells and the analog or derivative of the intermediary metabolite. If the least amount of regulatory, immune-regulatory or NKT cell proliferation is found in the fourth test tube, then that specific analog or derivative is a treatment for the disease.

In a preferred embodiment of the present invention, when administration takes place by oral means there is minimal interference with digestion and absorption of a mammalian intermediary metabolite, or an analog or derivative thereof, including but not limited to mammalian intermediary metabolites such as a lipid, a conjugated biomolecule, a polar lipid, a glycolipid, a glycosylceramide, lipoprotein, apolipoprotein, cytokine, or hormones, a monosaccharide ceramide, a glucosylceramide, a galactosylceramide, a disaccharide ceramide, a lactosylceramide, a sphingosine, a sphingolipid, a ceramide, a lipoprotein, an apolipoprotein, a cytokine, a hormone, a T cell ligand, a T cell receptor ligand, or a glycoprotein other than an antibody, in the mammalian subject. Specifically, the mammalian subject has been without food and/or water for a certain amount of hours prior to the administration of the aforesaid molecules, treatment of the mammalian subject or the manipulation of cells in the mammalian subject. When carrying out oral administration, the intermediary metabolite may be prepared synthetically or it may have been derived from a natural source; in the latter case the intermediary metabolite has undergone one or more purification steps to separate the intermediary metabolite from other substances that may have been present.

When treating cells ex vivo and readministering them to a patient, it is it is a subject of the present invention that non-mammalian intermediary metabolites may also find use in appropriate dosages. In a similar fashion, the screening method that has been described may also be used with non-mammalian intermediary metabolites. It is also a subject of the present invention that although a single intermediary metabolite may be used for treatment, there may also be benefits achieved by the use of a mixture that contains more than one intermediary metabolite.

1. A method for the treatment of a pulmonary, respiratory or airway disease or disorder in a mammalian subject comprising administering to said subject an effective amount of a one or more mammalian intermediary metabolites.

2. A method for the treatment of a pulmonary, respiratory or airway disease or disorder in a mammalian subject comprising administering to said subject an effective amount of one or more mammalian intermediary metabolites, wherein said mammalian intermediary metabolite is a T cell ligand or T cell receptor ligand.

3. A method for the treatment of a pulmonary, respiratory or airway disease or disorder in a mammalian subject comprising administering to said subject an effective amount of one or more mammalian intermediary metabolites, wherein said mammalian intermediary metabolite is a lipid, a polar lipid, or a conjugated biomolecule.

4. A method for the treatment of a pulmonary, respiratory or airway disease or disorder in a mammalian subject comprising administering to said subject an effective amount of one or more mammalian intermediary metabolites, wherein said mammalian intermediary metabolite is a glycolipid, lipoprotein, apolipoprotein, a glycoprotein other than an antibody, a cytokine or a hormone.

5. A method for the treatment of a pulmonary, respiratory or airway disease or disorder in a mammalian subject comprising administering to said subject an effective amount of one or more mammalian intermediary metabolites, wherein said mammalian intermediary metabolite is a monosaccharide ceramide, a disaccharide ceramide or a polysaccharide ceramide.

6. A method for the treatment of a pulmonary, respiratory or airway disease or disorder in a mammalian subject comprising administering to said subject an effective amount of one or more β-galactosylceramides, β-lactosylceramides, β-glucosylceramides, analogs of β-galactosylceramide other than an α-linked analog, β-galactosylceramide derivatives, analogs of β-lactosylceramide other than an α-linked analog, derivatives of β-lactosylceramide, analogs of β-glucosylceramide other than an α-linked analog, β-glucosylceramide derivatives, sulfated glycolipids, or any combination thereof.

7. The method of claim 1, 2, 3, 4, 5, or 6 wherein said disease is asthma.

8. An in vitro screening assay for an analog or derivative of a mammalian intermediary metabolite which is administered to a mammalian subject to treat a pulmonary, respiratory or airway disease or disorder comprising:
a) providing in vitro:
(i) regulatory, immune-regulatory or NKT cells from said subject or another subject;
(ii) antigen presenting cells; and
(iii) an analog or derivative of said mammalian intermediary metabolite; and
b) identifying a decrease in said regulatory, immune-regulatory or NKT cell proliferation.

9. An in vitro screening assay for an analog or derivative of a mammalian intermediary metabolite which is administered to a mammalian subject to treat a pulmonary, respiratory or airway disease or disorder comprising:
a) providing in vitro:
i) regulatory, immune-regulatory or NKT cells and BSA in a first test tube;
ii) regulatory, immune-regulatory or NKT cells and said analog or derivative of a mammalian intermediary metabolite in a second test tube;
iii) regulatory, immune-regulatory or NKT cells, antigen presenting cells and BSA in a third test tube; and
iv) regulatory, immune-regulatory or NKT cells, antigen-presenting cells and an analog or derivative of said mammalian intermediary metabolite;
b) determining the amount of regulatory, immune-regulatory or NKT cell proliferation in each of said tubes; and
c) identifying the least amount of regulatory, immune-regulatory or NKT cell proliferation in said fourth tube.

10. A method for treating a pulmonary, respiratory or airway disease or disorder in a mammalian subject comprising:
a) obtaining cells from said subject or another subject, said cells comprising regulatory, immune-regulatory or NKT cells;
b) treating or educating said cells ex vivo in the presence of:
i) one or more intermediary metabolites;
ii) one or more antigens or epitopes associated with said disease, or one or more antigens or epitopes associated with the immune-mediated inflammatory response;
iii) antigen presenting cells; or
iv) any combination of the above; and
c) re-administering to said subject said treated or educated cells.

11. A method for treating a pulmonary, respiratory or airway disease or disorder in a mammalian subject comprising:
a) obtaining cells from said subject or another subject, said cells comprising regulatory, immune-regulatory or NKT cells;
b) treating or educating said cells ex vivo in the presence of:
i) one or more intermediary metabolites;
ii) one or more antigens or epitopes associated with said disease, or one or more antigens or epitopes associated with the immune-mediated inflammatory response;
iii) antigen presenting cells; or
iv) any combination of the above;
c) re-administering to said subject said treated or educated cells; and
d) administering to said subject:
i) an effective amount of intermediary metabolite;
ii) antigen presenting cells;
iii) one or more antigens or epitopes associated with said disease, or one or more or more antigens or epitopes associated with the immune-mediated inflammatory response; or
iv) any combination of the above.

12. A method for the treatment of a pulmonary, respiratory or airway disease or disorder in a mammalian subject comprising administering to said subject:
a) an effective amount of one or more intermediary metabolites;
b) antigen presenting cells;
c) one or more antigens or epitopes associated with said disease, or one or more antigens or epitopes associated with the immune-mediated inflammatory response; or
d) any combination of the above.

13. A therapeutic composition for the treatment of a pulmonary, respiratory or airway disease or disorder in a mammalian subject comprising:
a) one or more intermediary metabolites;
b) antigen presenting cells;
c) one or more antigens or epitopes associated with said disease, or one or more antigens or epitopes associated with the immune-mediated inflammatory response; or
d) any combination of the above.

14. The in vitro screening assay of claim 8 or 9 wherein said intermediary metabolite comprises a T cell ligand, a T cell receptor ligand, a lipid, a polar lipid, a conjugated biomolecule, a glycolipid, a lipoprotein, an apolipoprotein, a glycoprotein other than an antibody, a cytokine, a hormone, a glycosylceramide, a glycosylceramide analog or derivative, a monosaccharide ceramide, a disaccharide ceramide, a polysaccharide ceramide, a lactosylceramide, a β-lactosylceramide, a lactosylceramide analog or derivative, a glucosylceramide, a β-glucosylceramide, a glucosylceramide analog or derivative, a galactosylceramide, a β-galactosylceramide, a galactosylceramide analog or derivative, a sulfated glycolipid, or any combination thereof.

15. The method of any one of claims 10 to 12 wherein said intermediary metabolite comprises a T cell ligand.

16. The method of any one of claims 10 to 12 wherein said intermediary metabolite comprises a T cell receptor ligand.

17. The method of any one of claims 10 to 12 wherein said intermediary metabolite comprises a lipid, a polar lipid, or a conjugated biomolecule.

18. The method of any one of claims 10 to 12 wherein said intermediary metabolite comprises a glycolipid, a sulfated glycolipid, a lipoprotein, an apolipoprotein, a glycoprotein other than an antibody, a cytokine, or a hormone.

19. The method of any one of claims 10 to 12 wherein said intermediary metabolite comprises a monosaccharide ceramide, a disaccharide ceramide or a polysaccharide ceramide.

20. The method of any one of claims 10 to 12 wherein said intermediary metabolite comprises a glucosylceramide, a lactosylceramide, or a galactosylceramide.

21. The method of any one of claims 10 to 12 wherein said intermediary metabolite comprises a galactosylceramide analog or derivative, a glucosylceramide analog or derivative or a lactosylceramide analog or derivative.

22. The therapeutic composition of claim 13 wherein said intermediary metabolite comprises a T cell ligand, a T cell receptor ligand, a lipid, a polar lipid, a conjugated biomolecule, a glycolipid, a lipoprotein, an apolipoprotein, a glycoprotein other than an antibody, a cytokine, a hormone, a glycosylceramide, a glycosylceramide analog or derivative, a monosaccharide ceramide, a disaccharide ceramide, a polysaccharide ceramide, a lactosylceramide, a β-lactosylceramide, a lactosylceramide analog or derivative, a glucosylceramide, a β-glucosylceramide, a glucosylceramide analog or derivative, a galactosylceramide, a β-galactosylceramide, a galactosylceramide analog or derivative, a sulfated glycolipid, or any combination thereof.

23. The method of any one of claims 10 to 12, or 15 to 21, wherein said one or more antigens comprise one or more allogeneic antigens obtained from donors suffering from said pulmonary, respiratory or airway disorder or disease, xenogenic antigens, syngeneic antigens, autologous antigens, non-autologous antigens, recombinantly prepared antigens, or any combination thereof.

24. The in vitro screening assay of claim 8, 9 or 14 wherein said one or more antigens comprise one or more allogeneic antigens obtained from donors suffering from said pulmonary, respiratory or airway disorder or disease, xenogenic antigens, syngeneic antigens, autologous antigens, non-autologous antigens, recombinantly prepared antigens, or any combination thereof.

25. The therapeutic composition of claim 13 or 22 wherein said one or more antigens comprise one or more allogeneic antigens obtained from donors suffering from said pulmonary, respiratory or airway disorder or disease, xenogenic antigens, syngeneic antigens, autologous antigens, non-autologous antigens, recombinantly prepared antigens, or any combination thereof.

26. The method of any one of claims 10 to 12 or 15 to 21 wherein said antigen presenting cell comprises a dendritic cell or a CD1d receptor-presenting dendritic cell.

27. The in vitro screening assay of any one of claims 8, 9, or 14 wherein said antigen presenting cell comprises a dendritic cell or a CD1d receptor-presenting dendritic cell.

28. The therapeutic composition of any one of claims 13 or 22 wherein said antigen presenting cell comprises a dendritic cell or a CD1d receptor-presenting dendritic cell.

29. The administering step of any one of claims 1 to 12, 14 to 21, 23, 24, 26 or 27 wherein said administration step comprises oral, intravenous, intraperitoneal, intramuscular, parenteral, transdermal, intravaginal, intranasal, mucosal, sublingual, topical, rectal or subcutaneous administration, or any combination thereof.

30. The method of any one of claims 1 to 6 and 8 to 29 wherein said disease is asthma.

31. A method for the treatment of a pulmonary, respiratory or airway disease or disorder in a mammalian subject comprising administering to said subject an effective amount of one or more mammalian intermediary metabolites, the result of said administration comprising a change in the number or function of regulatory, immune-regulatory or NKT cells.

32. A method for the treatment of a pulmonary, respiratory or airway disease or disorder in a mammalian subject comprising administering to said subject an effective amount of one or more mammalian intermediary metabolites, the result of said administration comprising the reduction, inhibition, or decrease of the number or function of regulatory, immune-regulatory or NKT cells.

33. A method for the treatment of a pulmonary, respiratory or airway disease or disorder in a mammalian subject comprising administering to said subject an effective amount of one or more mammalian intermediary metabolites, the result of said administration comprising the stimulation or increase of the number or function of regulatory, immune-regulatory or NKT cells.

34. The method of claim 31, 32 or 33 wherein said regulatory, immune-regulatory or NKT cells are intrahepatic NKT cells.

35. The method of claim 32 wherein said inhibition comprises the competitive displacement of activating elements from the CD1d molecule.

36. The method of claim 33 wherein said stimulation comprises the increased binding of activating elements from the CD1d molecule.

37. The method of claim 31, 32 or 33 wherein said result further comprises changes in cytokine responses.

38. The method of claim 37 wherein said cytokines comprise IFNγ, TNFα, IL2, IL4, IL10, or IL12.

39. The method of claim 37 wherein said cytokine response comprises a pro-inflammatory, anti-inflammatory or both a pro-inflammatory and anti-inflammatory response.

40. The method of claim 31, 32 or 33 wherein said result further comprises changes in the Th1/Th2 balance in said subject's immune system.

41. A method for the treatment of a pulmonary, respiratory or airway disease or disorder in a mammalian subject comprising administering to said subject an effective amount of one or more intermediary metabolites and antigen presenting cells, the result of said administration comprising a decrease in regulatory, immune-regulatory or NKT cell proliferation.

42. A method for the treatment of a pulmonary, respiratory, or airway disease or disorder in a mammalian subject comprising administering to said subject an effective amount of one or more intermediary metabolites and antigen presenting cells, the result of said administration comprising an increase in regulatory, immune-regulatory or NKT cell proliferation.

43. An in vitro screening assay for an analog or derivative of an intermediary metabolite which is administered to a mammalian subject to treat a pulmonary, respiratory or airway disease or disorder resulting in a change in the number of regulatory, immune-regulatory or NKT cells, said assay comprising:
a) providing in vitro:
(i) regulatory, immune-regulatory or NKT cells from said subject or another subject;
(ii) antigen presenting cells;
(iii) analog or derivative of said intermediary metabolite; and
b) identifying a decrease in said regulatory, immune-regulatory or NKT cell proliferation.

44. An in vitro screening assay for an analog or derivative of an intermediary metabolite which is administered to a mammalian subject to treat a pulmonary, respiratory or airway disease or disorder resulting in a change in the number of regulatory, immune-regulatory or NKT cells, said assay comprising:
a) providing in vitro:
i) regulatory, immune-regulatory or NKT cells and BSA in a first test tube;
ii) regulatory, immune-regulatory or NKT cells and said analog or derivative of an intermediary metabolite in a second test tube;
iii) regulatory, immune-regulatory or NKT cells, antigen presenting cells and BSA in a third test tube; and
iv) regulatory, immune-regulatory or NKT cells, antigen-presenting cells and an analog or derivative of said intermediary metabolite;
b) determining the amount of regulatory, immune-regulatory or NKT cell proliferation in each of said tubes; and
c) identifying the least amount of regulatory, immune-regulatory or NKT cell proliferation in said fourth tube.

45. A method for treating a pulmonary, respiratory or airway disease or disorder in a mammalian subject comprising:
a) obtaining cells from said subject or another subject, said cells comprising regulatory, immune-regulatory or NKT cells;
b) treating or educating said cells ex vivo in the presence of:
i) one or more intermediary metabolites;
ii) one or more antigens or epitopes associated with said disease, or one or more antigens or epitopes associated with the immune-mediated inflammatory response;
iii) antigen presenting cells; or
iv) any combination of the above; and
b) re-administering to said subject said treated or educated cells, the result of said administration comprising a change in the number of said cells.

46. A method for treating a pulmonary, respiratory or airway disease or disorder in a mammalian subject comprising:
a) obtaining cells from said subject or another subject, said cells comprising regulatory, immune-regulatory or NKT cells;
b) treating or educating said cells ex vivo in the presence of:
i) one or more intermediary metabolites;
ii) one or more antigens or epitopes associated with said disease, or one or more antigens or epitopes associated with the immune-mediated inflammatory response;
iii) antigen presenting cells; or
iv) any combination of the above;
c) re-administering to said subject said treated or educated cells;
d) administering to said subject:
i) an effective amount of one or more intermediary metabolites;
ii) antigen presenting cells;
iii) one or more antigens or epitopes associated with said disease, or one or more antigens or epitopes associated with the immune-mediated inflammatory response; or
iv) any combination of the above; and
e) the result of said administration comprising a change in the number of regulatory cells, immune-regulatory cells or NKT cells.

47. A method for the treatment of a pulmonary, respiratory or airway disease or disorder in a mammalian subject comprising administering to said subject:
a) an effective amount of one or more intermediary metabolites;
b) antigen presenting cells;
c) one or more antigens or epitopes associated with said disease, or one or more antigens or epitopes associated with the immune-mediated inflammatory response; or
d) any combination of the above;
e) the result of said administration comprising a change in the number of regulatory cells, immune-regulatory cells or NKT cells.

48. A method for treating a pulmonary, respiratory or airway disease or disorder in a mammalian subject comprising administering to said subject an effective amount of one or more mammalian intermediary metabolites so as to modulate or change at least one component in the immune system of said subject.

49. A therapeutic composition for the treatment of a pulmonary, respiratory or airway disease or disorder in a mammalian subject, the administration of said composition resulting in a change in the number of regulatory, immune-regulatory or NKT cells, said composition comprising:
a) one or more intermediary metabolites;
b) antigen presenting cells;
c) one or more antigens or epitopes associated with said disease; or one or more antigens or epitopes associated with the immune-mediated inflammatory response; or
d) any combination of the above.

50. The composition of claim 49 wherein said result comprises the reduction, inhibition, or decrease in the number or function of said cells.

51. The composition of claim 49 wherein said result comprises the stimulation or increase in the number or function of said cells.

52. The use of a mammalian intermediary metabolite in the manufacture of a composition for the manipulation of regulatory, immune-regulatory or NKT cells in a mammalian subject suffering from a pulmonary, respiratory or airway disease or disorder.

53. The method of claim 52 wherein said manipulation comprises a change in the number or function of said cells.

54. The method of claim 53 wherein said change comprises a reduction, inhibition or decrease of the number or function of said cells.

55. The method of claim 53 wherein said change comprises a stimulation or increase in the number or function of said cells.

56. The method of any one of claims 31 to 42, 45 to 48, or 52 to 55 wherein said intermediary metabolite or said mammalian intermediary metabolite comprises a T cell ligand.

57. The method of any one of claims 31 to 42, 45 to 48, or 52 to 55 wherein said intermediary metabolite or said mammalian intermediary metabolite comprises a T cell receptor ligand.

58. The method of any one of claims 31 to 42, 45 to 48, or 52 to 55 wherein said intermediary metabolite or said mammalian intermediary metabolite comprises a lipid, any polar lipid or conjugated biomolecule.

59. The method of any one of claims 31 to 42, 45 to 48, or 52 to 55 wherein said intermediary metabolite or said mammalian intermediary metabolite comprises a glycolipid, a lipoprotein, an apolipoprotein or a glycoprotein other than antibodies, cytokines, or hormones.

60. The method of any one of claims 31 to 42, 45 to 48, or 52 to 55 wherein said intermediary metabolite or said mammalian intermediary metabolite comprises a monosaccharide ceramide, a disaccharide ceramide or a polysaccharide ceramide.

61. The method of any one of claims 31 to 42, 45 to 48, or 52 to 55 wherein said intermediary metabolite or said mammalian intermediary metabolite comprises a glucosylceramide, a galactosylceramide or a lactosylceramide.

62. The method of any one of claims 31 to 42, 45 to 48, or 52 to 55 wherein said intermediary metabolite or said mammalian intermediary metabolite comprises a glucosylceramide, a β-galactosylceramide or a β-lactosylceramide.

63. The method of any one claims 31 to 42, 45 to 48, or 52 to 55 wherein said intermediary metabolite comprises a β-glucosylceramide analog or derivative, a β-galactosylceramide analog or derivative or a β-lactosylceramide analog or derivative.

64. The in vitro screening assay of claims 43 or 44 wherein said intermediary metabolite comprises a T cell ligand, a T cell receptor ligand, a lipid, a polar lipid, a conjugated biomolecule, a glycolipid, a lipoprotein, an apolipoprotein, a glycoprotein other than an antibody, a cytokine, a hormone, a glycosylceramide, a glycosylceramide analog or derivative, a monosaccharide ceramide, a disaccharide ceramide, a polysaccharide ceramide, a lactosylceramide, a β-lactosylceramide, a lactosylceramide analog or derivative, a glucosylceramide, a β-glucosylceramide, a glucosylceramide analog or derivative, a galactosylceramide, a β-galactosylceramide, a galactosylceramide analog or derivative, a sulfated glycolipid, or any combination thereof.

65. The therapeutic composition of any one of claims 49 to 51 wherein said intermediary metabolite comprises a T cell ligand, a T cell receptor ligand, a lipid, a polar lipid, a conjugated biomolecule, a glycolipid, a lipoprotein, an apolipoprotein, a glycoprotein other than an antibody, a cytokine, a hormone, a glycosylceramide, a glycosylceramide analog or derivative, a monosaccharide ceramide, a disaccharide ceramide, a polysaccharide ceramide, a lactosylceramide, a β-lactosylceramide, a lactosylceramide analog or derivative, a glucosylceramide, a β-glucosylceramide, a glucosylceramide analog or derivative, a galactosylceramide, a β-galactosylceramide, a galactosylceramide analog or derivative, a sulfated glycolipid, or any combination thereof.

66. The method of any one of claims 41 to 42, 45 to 48, or 52 to 63 wherein said antigens comprise allogeneic antigens obtained from donors suffering from said pulmonary, respiratory or airway disease or disorder, xenogenic antigens, syngeneic antigens, autologous antigens, non-autologous antigens, recombinantly prepared antigens, or any combination thereof.

67. The in vitro screening assay of any one of claims 43, 44 or 64 wherein said antigens comprise allogeneic antigens obtained from donors suffering from said pulmonary, respiratory or airway disease or disorder, xenogenic antigens, syngeneic antigens, autologous antigens, non-autologous antigens, recombinantly prepared antigens, or any combination thereof.

68. The therapeutic composition of any one of claims 49, 50, 51 or 65 wherein antigens comprise allogeneic antigens obtained from donors suffering from said pulmonary, respiratory or airway disease or disorder, xenogenic antigens, syngeneic antigens, autologous antigens, non-autologous antigens, recombinantly prepared antigens, or any combination thereof.

69. The method of any one of claims 41 to 42, 45 to 48, 52 to 63, or 66 wherein said antigen presenting cell comprises a dendritic cell or a CD1 receptor-presenting dendritic cell.

70. The in vitro screening assay of any one of claims 43, 44, 64 or 67 wherein said antigen presenting cell comprises a dendritic cell or a CD1 receptor-presenting dendritic cell.

71. The therapeutic composition of any one of claims 49, 50, 51, 65 or 68 wherein said antigen presenting cell comprises a dendritic cell or a CD1 receptor-presenting dendritic cell.

72. The method of any one of claims 31 to 42, 45 to 48, 52 to 63, 66 or 69 wherein said disease is asthma.

73. The in vitro screening assay of any one of claims 43, 44, 64, 67 or 70 wherein said disease is asthma.

74. The therapeutic composition of any one of claims 49, 50, 51, 65, 68 or 71 wherein said disease is asthma.

75. The administering step of any one of claims 31 to 51, or 56 to 74 wherein said administration comprises oral, intravenous, intraperitoneal, intramuscular, parenteral, transdermal, intravaginal, intranasal, mucosal, sublingual, topical, rectal or subcutaneous administration, or any combination thereof.

76. A method for the treatment of a pulmonary, respiratory or airway disease or disorder in a mammalian subject comprising administering to said subject an effective amount of a mammalian intermediary metabolite, the result of said administration comprising an alteration of the regulatory, immune-regulatory or NKT cell distribution in said subject.

77. A method for the treatment of a pulmonary, respiratory or airway disease or disorder in a mammalian subject comprising administering to said subject an effective amount of an intermediary metabolite, the result of said administration comprising an alteration of the regulatory, immune-regulatory or NKT cell distribution in said subject and/or a change in the peripheral/intrahepatic T cell ratio.

78. The method of claim 77 wherein said change in the peripheral/intrahepatic T cell ratio comprises an increase in said ratio.

79. A method for the treatment of a pulmonary, respiratory or airway disease or disorder in a mammalian subject comprising administering to said subject an effective amount of a mammalian intermediary metabolite, the result of said administration comprising an alteration of the regulatory, immune-regulatory or NKT cell distribution in said subject and/or a change in intrahepatic CD8+ T cell trapping.

80. The method of claim 79 wherein said change in intrahepatic CD8+ T cell trapping comprises an increase in said trapping.

81. The method of any one of claims 76 to 80 wherein said regulatory, immune-regulatory or NKT cells comprise intrahepatic or intrasplenic NKT cells.

82. The method of any one of claims 76 to 80 or wherein said result further comprises changes in cytokine responses.

83. The method of claim 82 wherein said cytokines comprise IFNγ, TNFα, IL2, IL4, IL10 or IL12.

84. The method of claim 82 wherein said cytokine response comprises a pro-inflammatory, anti-inflammatory or both a pro-inflammatory and anti-inflammatory response.

85. The method of any one of claims 76 to 80 wherein said result further comprises changes in the Th1/Th2 balance in said subject's immune system.

86. A method for the treatment of a pulmonary, respiratory or airway disease or disorder in a mammalian subject comprising administering to said subject an effective amount of an intermediary metabolite and/or antigen presenting cells, the result of said administration comprising an alteration of the regulatory, immune-regulatory or NKT cell distribution.

87. A method for the treatment of a pulmonary, respiratory or airway disease or disorder in a mammalian subject comprising administering to said subject an effective amount of an intermediary metabolite and/or antigen presenting cells, the result of said administration comprising an alteration of the function of the regulatory, immune-regulatory or NKT cell distribution.

88. An in vitro screening assay for an analog or derivative of an intermediary metabolite which is administered to a mammalian subject to treat a pulmonary, respiratory or airway disease or disorder resulting in an alteration of the regulatory, immune-regulatory or NKT cell distribution, said assay comprising:
a) providing in vitro:
(i) regulatory, immune-regulatory or NKT cells from said subject or another subject;
(ii) antigen presenting cells;
(iii) analog or derivative of said intermediary metabolite; and
b) identifying a decrease in said regulatory, immune-regulatory and NKT cell proliferation.

89. An in vitro screening assay for an analog or derivative of an intermediary metabolite which is administered to a mammalian subject to treat a pulmonary, respiratory or airway disease or disorder resulting in an alteration of the regulatory, immune-regulatory or NKT cell distribution, said assay comprising:
a) providing in vitro:
i) regulatory, immune-regulatory or NKT cells and BSA in a first test tube;
ii) regulatory, immune-regulatory or NKT cells and said analog or derivative of an intermediary metabolite in a second test tube;
iii) regulatory, immune-regulatory or NKT cells, antigen presenting cells and BSA in a third test tube;
iv) regulatory, immune-regulatory or NKT cells, antigen-presenting cells and an analog or derivative of said intermediary metabolite;
b) determining the amount of regulatory, immune-regulatory or NKT cell proliferation in each of said tubes; and
c) identifying the least amount of regulatory, immune-regulatory or NKT cell proliferation in said fourth tube.

90. A method for treating a pulmonary, respiratory, or airway disease or disorder in a mammalian subject comprising:
a) obtaining cells from said subject or another subject, said cells comprising regulatory, immune-regulatory or NKT cells;
b) treating or educating said cells ex vivo in the presence of:
i) intermediary metabolite;
ii) antigens or epitopes associated with said disease, or antigens or epitopes associated with the immune-mediated inflammatory response;
iii) antigen presenting cells; or
iv) any combination of the above;
b) re-administering to said subject said treated or educated cells, the result of said administration comprising an alteration in said cell distribution.

91. A method for treating a pulmonary, respiratory or airway disease or disorder in a mammalian subject comprising:
a) obtaining cells from said subject or another subject, said cells comprising regulatory, immune-regulatory or NKT cells;
b) treating or educating said cells ex vivo in the presence of:
i) one or more intermediary metabolites;
ii) one or more antigens or epitopes associated with said disease or disorder, or one or more antigens or epitopes associated with an immune-mediated inflammatory response;
iii) antigen presenting cells; or
iv) any combination of the above;
c) re-administering to said subject said treated or educated cells; and
d) administering to said subject:
i) an effective amount of one or more intermediary metabolites;
ii) antigen presenting cells;
iii) one or more antigens or epitopes associated with said disease, or one or more antigens or epitopes associated with the immune-mediated inflammatory response; or
iv) any combination of the above; and
e) the result of said administration comprising an alteration of the regulatory, immune-regulatory or NKT cell distribution.

92. A method for the treatment of a pulmonary, respiratory or airway disease or disorder in a mammalian subject comprising administering to said subject:
a) an effective amount of an intermediary metabolite;
b) antigen presenting cells;
c) one or more antigens or epitopes associated with said disease or disorder, or one or more antigens or epitopes associated with the immune-mediated inflammatory response; or
d) any combination of the above; and
e) the result of said administration comprising an alteration of the regulatory, immune-regulatory or NKT cell distribution.

93. A method for treating a pulmonary, respiratory or airway disease or disorder in a mammalian subject comprising administering to said subject an effective amount of a mammalian intermediary metabolite so as to modulate or change at least one component in the immune system of said subject.

94. A therapeutic composition for the treatment of a pulmonary, respiratory or airways disease or disorder in a mammalian subject, the administration of said composition resulting in an alteration of the regulatory, immune-regulatory or NKT cell distribution, said composition comprising:
a) one or more intermediary metabolites;
b) antigen presenting cells;
c) one or more antigens or epitopes associated with said disease or disorder; or one or more antigens or epitopes associated with the immune-mediated inflammatory response; or
d) any combination of the above.

95. The use of a mammalian intermediary metabolite in the manufacture of a composition for the manipulation of regulatory, immune-regulatory or NKT cells in a mammalian subject suffering from a pulmonary, respiratory or airway disease or disorder.

96. The method of claim 95 wherein said manipulation comprises a change of the distribution of said cells in said subject.

97. The method of any one of claims 76 to 87, 90 to 93, 95 or 96 wherein said intermediary metabolite comprises a T cell ligand, a T cell receptor ligand, a lipid, a polar lipid, a conjugated biomolecule, a glycolipid, a lipoprotein, an apolipoprotein, a glycoprotein other than an antibody, a cytokine, a hormone, a glycosylceramide, a glycosylceramide analog or derivative, a monosaccharide ceramide, a disaccharide ceramide, a polysaccharide ceramide, a lactosylceramide, a β-lactosylceramide, a lactosylceramide analog or derivative, a glucosylceramide, a β-glucosylceramide, a glucosylceramide analog or derivative, a galactosylceramide, a β-galactosylceramide, a galactosylceramide analog or derivative, a sulfated glycolipid, or any combination thereof.

98. The in vitro assay of claims 88 to 89 wherein said intermediary metabolite comprises a T cell ligand, a T cell receptor ligand, a lipid, a polar lipid, a conjugated biomolecule, a glycolipid, a lipoprotein, an apolipoprotein, a glycoprotein other than an antibody, a cytokine, a hormone, a glycosylceramide, a glycosylceramide analog or derivative, a monosaccharide ceramide, a disaccharide ceramide, a polysaccharide ceramide, a lactosylceramide, a β-lactosylceramide, a lactosylceramide analog or derivative, a glucosylceramide, a β-glucosylceramide, a glucosylceramide analog or derivative, a galactosylceramide, a β-galactosylceramide, a galactosylceramide analog or derivative, a sulfated glycolipid, or any combination thereof.

99. The therapeutic composition of claim 94 wherein said intermediary metabolite comprises a T cell ligand, a T cell receptor ligand, a lipid, a polar lipid, a conjugated biomolecule, a glycolipid, a lipoprotein, an apolipoprotein, a glycoprotein other than an antibody, a cytokine, a hormone, a glycosylceramide, a glycosylceramide analog or derivative, a monosaccharide ceramide, a disaccharide ceramide, a polysaccharide ceramide, a lactosylceramide, a β-lactosylceramide, a lactosylceramide analog or derivative, a glucosylceramide, a β-glucosylceramide, a glucosylceramide analog or derivative, a galactosylceramide, a β-galactosylceramide, a galactosylceramide analog or derivative, a sulfated glycolipid, or any combination thereof.

100. The method of any one of claims 90, 91, 92 or 94 wherein said antigens comprise allogeneic antigens obtained from donors suffering from said immune-related or immune-mediated disorder or disease, xenogenic antigens, syngeneic antigens, autologous antigens, non-autologous antigens, recombinantly prepared antigens, or any combination thereof.

101. The method of any one of claims 86, 87, 88, 89, 90, 91, 92, or 94 wherein said antigen presenting cell comprises a dendritic cell or a CD1d receptor-presenting dendritic cell.

102. The method of any one of claims 76 to 101 wherein said pulmonary, respiratory or airway disease or disorder comprises asthma.

103. The method of any one of claims 76 to 101 wherein said administering step comprises oral, intravenous, intraperitoneal, intramuscular, parenteral, transdermal, intravaginal, intranasal, mucosal, sublingual, topical, rectal or subcutaneous administration, or any combination thereof.

104. The in vitro assay of any one of claims 88, 89, or 98 wherein said administering step comprises oral, intravenous, intraperitoneal, intramuscular, parenteral, transdermal, intravaginal, intranasal, mucosal, sublingual, topical, rectal or subcutaneous administration, or any combination thereof.

105. The therapeutic composition of claim 94 or 99 wherein said administering step comprises oral, intravenous, intraperitoneal, intramuscular, parenteral, transdermal, intravaginal, intranasal, mucosal, sublingual, topical, rectal or subcutaneous administration, or any combination thereof.

106. The method of any one of claims 1 to 104 wherein said mammalian subject has been without food and/or water for a minimum of twelve hours prior to said administration, treatment or manipulation.

107. The method of any one of claims 1 to 104 wherein said mammalian subject has been subjected to fasting for a minimum of twelve hours prior to said administration, treatment or manipulation.

108. The method of claim 103, 104 or 105 wherein said orally administered intermediary metabolite is in a purified form.

109. The method of any one of claims 1 to 108 wherein said intermediary metabolite is prepared synthetically or is derived from a non-mammalian source.
